(19) United States
(12) Patent Application Publication
(10) Pub. No.: US 2017/0189316 A1
(43) Pub. Date: Jul. 6, 2017

(54) HYBRID NAIL COATING SYSTEMS AND METHODS OF THEIR USE

(71) Applicant: Nail Alliance, LLC, Gladstone, MO (US)

(72) Inventor: Sunil Sirdesai, Irvine, CA (US)

(21) Appl. No.: 15/396,207

(22) Filed: Dec. 30, 2016

Related U.S. Application Data
(60) Provisional application No. 62/273,655, filed on Dec. 31, 2015.

(51) Int. Cl. A61K 8/892; A61K 8/37; A61K 8/34; A61K 8/73; A61K 8/85; A61K 8/58; A61K 8/40; A61K 8/81; A61K 8/46; A61K 8/891; A61K 8/29; A61K 8/49; A61K 8/64; A61K 8/87; A61K 8/25; A45D 29/00; A61Q 3/02

(52) U.S. Cl. CPC A61K 8/892; A61Q 3/02; A61K 8/37; A61K 8/34; A61K 8/731; A61K 8/85; A61K 8/58; A61K 8/40; A61K 8/8152; A61K 8/46; A61K 8/891; A61K 8/29; A61K 8/498; A61K 8/645; A61K 8/347; A61K 8/87; A61K 8/25; A45D 29/00; A61K 2800/81

(57) ABSTRACT

Novel hybrid coating compositions for mammalian nails, systems comprising the compositions, kits containing these compositions or systems, and methods of their use are disclosed. The novel compositions and/or kits are useful, inter alia, for providing durable, “soak-off” type nail coatings.

### FIELD OF THE INVENTION

The present invention relates generally to hybrid nail coating compositions, kits containing these compositions, and methods of their use and preparation.

### BACKGROUND OF THE INVENTION

The information provided below is not admitted to be prior art to the present invention, but is provided solely to assist the understanding of the reader.

Consumers use nail coatings to cosmetically enhance the appearance of their nails or protect the nails from the abuses found in their everyday environment. However, nail coating compositions typically lack the durability desired by consumers or are difficult to apply or remove in terms of time and/or effort. The lack of durability is often evidenced by a chipping or peeling of the coating soon after the original coating has been applied, requiring at least in part a reapplication of the coating in an attempt to recreate the aesthetic appearance or the therapeutic benefits of the original nail coating. Application and/or removal of more durable nail coatings is often very time consuming, requiring long cure times upon administration by skilled personnel leading to added costs for the consumer.

Light curable nail coatings are disclosed in Billings, U.S. Pat. No. 5,194,292, entitled “Method of Drying and Bonding Nail Polish”; Cornell, U.S. Pat. No. 4,704,303, entitled “Nail Extension Composition”; and Guiliano, U.S. Pat. No. 4,682,612, entitled “Novel Process and Article for Preparing Artificial Nails”. The '292 Patent is reportedly describes a method of protecting common nail polish by applying a light-curable clear coating over the polished nail. The '303 Patent reportedly describes a coating composition based on an aliphatic or cycloaliphatic hydrocarbon urethane diacrylate or methacrylate having a molecular weight of 250 to 500 and a viscosity of 5,000 to 30,000 cps. Cornell reports that radiation in the visible region is used to cure the '303 Patent coatings. The '612 Patent describes an organic solvent-free photo-curable composition which has at least one liquid monomer in which an acrylated urethane oligomer is dissolved and cross-linked upon cure. Giuliano reports that radiation in the UV region is used to cure the '612 Patent coatings.

Lilley (U.S. Pat. Nos. 6,391,938, 6,803,394 and 6,599,958) discloses light cured nail coatings that are applied to natural nails and/or artificial nail tips for cosmetic purposes.

Ellingson et al. (U.S. Pat. No. 6,306,375) discloses long wear nail polish compositions having defined surface properties, as well as kits, films and methods of their use.

Ellingson et al. (U.S. Pat. No. 6,123,931) discloses polyurethane and polyacryl nail polish compositions useful as coatings for mammalian nails as well as methods of their use.

Ellingson et al. (U.S. Pat. No. 6,136,300) discloses long wear nail polish compositions having adhesion, toughness and hardness characteristics useful as coatings for mammalian nails as well as methods of their use.

Smith III et al. (U.S. Pat. No. 6,080,414) discloses films and kits useful as polishes for mammalian nails and methods of their use, reportedly having long wear characteristics.

Farer et al. (U.S. Pat. No. 6,656,483) discloses cosmetic compositions containing polyurethane for application to the skin and nails.

Farer et al. (U.S. Pat. No. 6,156,325) and Carrion et al. (U.S. Pat. No. 6,555,096 and related published U.S. Patent Application No. 2002/0102222) disclose nail enamel compositions containing a urea-modified thixotropic agent.

Sirdesai et al. (U.S. Pat. No. 6,244,274) discloses certain polymerizable thixotropic oligomeric compositions for sculpting artificial fingernails which is non-yellowing, and which maintains its shape when formed and polymerizes rapidly under actinic radiation.

Sirdesai et al. (U.S. Pat. No. 5,785,958) discloses certain rapid drying top coat used to provide a durable glossy look to manicured nails.

Montgomery et al. (U.S. Pat. No. 4,766,005) describes materials and methods for obtaining strong adhesive bonds of certain coatings to keratin substrates.

Pagano et al. (U.S. Pat. No. 5,772,988) discloses nail enamel compositions comprising solvent and certain copolymers.

Kozachek et al. disclose certain UV curable, thixotropic, radiation curable, low viscosity gels comprised of a formulation containing thixotropic additive(s) and in some cases dispersants for additional dispersion stability to reportedly prolong shelf life and long-time storage at ambient conditions. (U.S. Published Patent Application Ser. Nos. 2011/0256079 and 2011/0256080.

Certain adhesion-promoting curable basecoat formulations are disclosed by Conger et al. (U.S. Pat. No. 8,263,677). Certain artificial nail color coats that may be employed with the basecoat formulations are disclosed in Vu et al. (U.S. Pat. no. 8,492,454). Certain reportedly scratch-resistant and protective top coat layers for these same compositions that can be easily removed are disclosed in Vu et al. (U.S. Pat. No. 8,541,482).

Conventional nail coatings may be classified into two categories: nail polishes; also known as lacquers, varnish or enamels; and artificial nails, also referred to as gels or acrylics. Nail polishes typically comprise various preformed polymer components (polymeric film forming agents) which are dissolved and/or suspended in non-reactive solvents. Upon application and drying, the solids deposit on the nail surface as a clear, translucent or colored film. Typically, nail polishes are easily scratched and are readily removable with solvent, usually within one minute and, if not removed as described, tend to chip or peel from the natural nail in one to five days.

Artificial nails including, for example, powder/liquid systems and gel systems, do not form films by evaporation, like polishes. Rather, they typically contain large amounts of reactive olefins (monomeric, oligomeric, and/or polymeric) that are susceptible to free radical polymerization. When radical initiators are present and chemically or photochemically activated, they set off a chain reaction forming polymeric chains that form the basis of the film former in the applied composition. These compositions may also include polymeric film formers similar to those used in nail polishes. While these systems provide more durability than polishes, they tend to be much more difficult or time consuming to apply and/or remove than their counterpart polishes. A further problem in known light curable nail coatings is “leftover” photoinitiator by-products formed by photopolymerization. These by-products can cause yellowing of the coating and risk skin sensitization in the general population. A coating is needed which comprises a reduced amount of photoinitiators to reduce yellowing and potentially harmful skin sensitization.

Conventional artificial nails are comprised of chemically reactive monomers, and/or oligomers, in combination with reactive or non-reactive polymers to create systems which are typically 100% solids and do not require non-reactive solvents. Upon pre-mixing and subsequent application to the nail plate, or application and exposure to UV radiation, a chemical reaction ensues resulting in the formation of long-lasting, highly durable cross-linked thermoset nail coating that is difficult to remove. Artificial nails may possess greatly enhanced adhesion, durability, as well as scratch and solvent resistance when compared to nail polishes. However, because of these inherent properties, such thermosets are much harder to remove, should the consumer so desire. Removal typically requires soaking in non-reactive solvents for 15-90 minutes (for acrylics and currently available “soakable gels”; it may take more than 90 minutes if ever to remove traditional UV nail gels by solvent) and typically may also require heavily abrading the surface or scraping with a wooden or metal probe to assist the removal process. Many acrylic systems do not contain any non-reactive solvents because reactive monomers in the compositions also act as diluents to keep the systems mobile for the sake of application.

Gel systems, in contrast to the traditional polish and other polymer-type systems, particularly ultraviolet-cured gel systems, often comprise a gel that may be brushed onto the nails, cured, and shaped to create artificial nails. Gel systems, as compared with traditional polishes have reduced solvent odor, are more durable and provide reasonable shine. They are generally more expensive, remain more difficult to remove and/or require specialized equipment such as curing lamps to prepare the nail coating. As compared with other polymer systems, they may be less durable and often require skilled personnel to apply them.

While thicker nail coatings may in general be more desirable due to their richer color and/or greater durability of the finished nail coating, it can be challenging to reasonably rapidly and substantially cure the entirety of the coating after its application. This is especially true for thicker and/or more highly pigmented UV-curable gel-based nail coating systems. This may be due, in part, to the nature of these coatings. For example, while UV light may readily penetrate the outermost regions of the coating composition to initiate the cure, the higher levels and/or darker hues of certain pigments in some coating compositions may limit penetration of the UV radiation into the innermost regions of the applied gel coating composition and thus increase the time required to substantially cure the entirety of the coating.

Often there is also a trade off in the choice of nail coatings between a particular coating's durability and its ease of removal. For example, some prior art gel coating compositions, while durable, cannot be readily removed by typical “soak-off” procedures and require a more laborious removal process. Alternatively, while some prior art gel coating compositions are very easily removed; those properties may lead to premature chipping and/or peeling of the coating, requiring additional maintenance or reapplication to stabilize the coating's overall appearance. Each type of artificial nail coating also benefits from application by skilled personnel.

Thus, a need exists for a composition colored or not, which is (are) easily applied, dries rapidly, does not yellow or cause skin sensitization in the general population, protects the nail more than lacquer polishes, lasts longer or is more durable that typical lacquer nail polishes, and can be removed when the wearer desires, including multi-layer systems comprising such compositions. Such compositions and multi-layer systems are described in embodiments of the present invention. A need also exists in the art for nail coating systems that can be formulated to provide durable curable nail coatings, especially those capable of being removed relatively easily by “soak-off” procedures. There is also a need for nail coating systems that are capable of being applied easily and/or without specialized equipment and/or in less time than typically required by prior art artificial nail systems. Further, there is a need for nail coatings that do not require curing after each layer is applied. There is, moreover, a need to provide coatings that may be applied similarly to polishes and yet improve durability of the finished coating. Additionally, there is a need for systems that provide such coatings regardless of the required coating color while providing a richness of color throughout the nail coating. Such coatings may give a more appealing and defect free appearance. Polishes that more readily adhere to the nail surface, especially those that provide a polymeric network connecting nail surface adhesion enhancing agents with outer layer film forming agents and/or those that are more durable or more readily removed when desired, are still needed. The present invention is directed to these and other important ends. Other objects and advantages will become apparent from the following disclosure.

### SUMMARY OF THE INVENTION

Accordingly, the present invention is directed, in part, to hybrid nail coating compositions, comprising a solvent, a film former, and a photoinitiator, the photoinitiator comprising a self-initiating oligomer.

The present invention is also directed, in part, to hybrid nail coating systems comprising a first layer comprising a solvent, a film former, and a photoinitiator, the photoinitiator comprising a self-initiating oligomer.

In certain other embodiments, the present invention is directed to kits suitable for coating mammalian nails containing hybrid nail coating compositions or hybrid nail coating systems containing such hybrid nail coating compositions, the hybrid nail coating compositions comprising a solvent, a film former, and a photoinitiator, the photoinitiator comprising a self-initiating oligomer; and a bottle for containing hybrid nail coating compositions, the bottle designed to substantially exclude the passage of UV light.

In some embodiments, the present invention is directed to methods of coating mammalian nails with a hybrid nail coating composition, comprising a solvent, a film former, and a photoinitiator, the photoinitiator comprising a self-initiating oligomer, wherein the method comprises (1) applying a nail coating composition according to the present invention contiguously to a mammalian nail or previously applied nail coating composition; and (2) curing the composition on the nail or nail coating.

### DETAILED DESCRIPTION OF THE PREFERRED EMBODIMENTS

As used herein, the term “non-aqueous nail coating composition” refers to a nail coating composition having no more than a de minimis quantity of water.

As used herein, “alkylene” refers to a saturated straight chain or branched hydrocarbon diradical having from about 2 to about 15 carbon atoms (and all combinations and subcombinations of ranges and specific numbers of carbon atoms therein), preferably with from about 4 to about 12, more preferably 6 to about 10, yet more preferably about 7 to about 9, with about 8 to about 9 carbon atoms being most preferred. Alkylene groups include, but are not limited to, ethylene, n-propylene, methylethylene, dimethylmethylene, n-butylene, isobutylene, dimethylethylene, methylpropylene, ethylethylene, n-pentylene, isopentylene, neopentylene, trimethylethylene, dimethylpropylene, methylbutylene, ethylpropylene, n-hexylene, isohexylene, neo-hexylene, methylpentylene, dimethylbutylene, and trimethylpropylene, methylethylpropylene, n-heptylene, isoheptylene, neo-heptylene, dimethylpentylene, ethylpentylene, trimethylbutylene, methylethylbutylene, n-octylene, isooctylene, neo-octylene, methyl heptylene, dimethylhexylene, trimethylpentylene, methylethylpentylene, n-nonylene, isononylene, neo-nonylene, methyloctylene, dimethylheptylene, trimethylhexylene, methylethylhexylene, trimethylheptylene, methylethylheptylene, n-decylene, isodecylene, neo-decylene, methylnonylene, dimethyloctylene, trimethylheptylene, methylethylheptylene, trimethyloctylene, methylethyloctylene, and tetramethylhexylene.

As used herein, “polyurethane acrylate oligomer” refers to polyurethane monomers, oligomers, or polymers, and mixtures thereof wherein the acrylate portion of the polyurethane acrylate oligomer is derived from one or more hydroxyalkylacrylic acid esters or hydroxyalkylmethacrylic acid esters, preferably hydroxyalkylmethacrylic acid esters. Non-limiting examples of hydroxyalkylmethacrylic acid esters include hydroxyethylmethacrylic acid ester and hydroxypropylmethacrylic acid ester. The structural backbone of the monomeric, oligomeric, and/or polyurethane acrylates and/or methacrylates is typically based on an alkylene or cycloalkylene moiety and may be derived from an alkylene or cycloalkylene isocyanate.

As used herein, “(meth)acrylated urethanes” refer to monomeric, oligomeric, and/or polyurethane acrylates and/or methacrylates having a structural urethane backbone that may be derived from an alkylene or cycloalkylene isocyanate derivable respectively from an alkylene or cycloalkylene diamine, the isocyanate including but not limited to trimethylhexylene diisocyanate. Other alkylene moieties are defined herein. Preferably, the (meth)acrylated urethane, more preferably methacrylated urethane, is derived by reaction of a hydroxyalkyl ester, preferably hydroxyethyl, more preferably 2-hydroxyethylester or hydroxypropylester, preferably 3-hydroxypropylester, or combination thereof, of an acrylic acid, preferably methacrylic acid that has been reacted with trimethylhexylene diisocyanate. The (meth)acrylated urethanes have at least some terminal (meth)acrylate moieties capable of further reacting to form oligomers or polymers with other olefinically unsaturated compounds present in any of the composition or system layers when acted upon by or in the presence of free-radical initiators. Exemplary cycloalkylene diisocyanates include isophorone diisocyanate. Exemplary “(meth)acrylated urethanes” include, any monomeric, oligomeric or polymeric form and mixtures thereof the following: di-HEMA trimethylhexyl dicarbamate, di-HPMA trimethylhexyl dicarbamate; (HEMA)(HPMA) trimethylhexyl dicarbamate, di-HEA trimethylhexyl dicarbamate, di-HPA trimethylhexyl dicarbamate; (HEA)(HPA) trimethylhexyl dicarbamate, di-HEMA isophorone dicarbamate, di-HPMA isophorone dicarbamate; (HEMA)(HPMA) isophorone dicarbamate, di-HEA isophorone dicarbamate, di-HPA isophorone dicarbamate; and (HEA)(HPA) isophorone dicarbamate.

The compositions, systems, kits containing such compositions, and methods of use and/or preparation of such compositions, systems, and/or kits of the present invention are directed in part to meet a need in the industry for colored or substantially clear coating compositions with improved properties as compared to prior art nail lacquer-type coatings. Thus, in certain embodiments, the present invention provides nail coating compositions that are removable using typical “soak-off” procedures, i.e., compositions that may be broken down and removed with solvents readily available for such purpose, including for example, acetone and/or other ketones, short chain alcohols, such as isopropanol, diacetone alcohol, C₁-C₈ alcohols, and the like, acrylic removers, tip removers, and/or various other acetate solvents, or any combination thereof. This invention has industrial applicability in providing compositions and methods for improving the adhesion of nail coatings to natural nails without requiring abrasion of the natural nail. The invention further provides means for removing a nail coating without requiring extended soak times or abrasion of the natural nail surface.

In other embodiments of the present invention, there are provided nail coating compositions that are applied in a fashion analogous to typical solvent-based lacquers, where solvent(s) within the coating composition substantially evaporate from the composition subsequent to its application, leaving any residual polymeric components contained in the composition to form a film on the nail or previously applied coating. Unlike typical lacquer-type nail coating compositions, the present compositions contain reactive components in addition to solvents and polymeric film formers. These reactive components, upon exposure to light or other radical initiator, react to form a matrix capable of providing further strength and/or durability to the nail coating or film being formed or deposited on the nail.

I. Hybrid Nail Coating Compositions

In certain embodiments, the present invention is directed, in part, to a hybrid nail coating composition, comprising a solvent, a film former, and a photoinitiator, the photoinitiator comprising a self-initiating oligomer.

A. Solvent

In certain aspects, the composition comprises a solvent. The choice of solvent is not critical, so long as the solvent does not substantially interfere with the irradiation and/or set or curing of the coating. The solvent may comprise a single component or may be a mixture of solvents. Typically the solvent is substantially non-aqueous. Preferably, the solvent is non-aqueous. In certain aspects, the solvent or solvents are cosmetically acceptable. By way of example, the solvents may include compounds such as esters, ketones, alcohols, alkanes, aromatics, and amides, preferably esters, ketones, and/or alcohols. In certain more preferred embodiments, the solvent is selected from the group consisting of butyl acetate, ethyl acetate, propyl acetate, isobutyl acetate, ethanol, isopropyl alcohol, butyl alcohol, amyl acetate, acetone, 2-butanone, and diacetone alcohol, trimethylpentanyl diisobutyrate, and mixtures thereof. The combined weight percentage of solvent or solvents in the nail coating compositions are such that the solvent is typically present at a range of from about 50% to about 90%, preferably from about 60% to about 90%, more preferably about 60% to about 80%, still more preferably of from about 61%, 62%, 63%, 64%, or 65% to about 80% by weight of the nail coating composition (and all combinations and subcombinations of ranges of solvents therein).

In certain other aspects, the hybrid nail coating compositions of the present invention include a butyl acetate solvent (for example, n-butyl, isobutyl, or secondary butyl acetate or any combination thereof), preferably butyl acetate having INCI designation, “49”. The butyl acetate solvent is typically present at a range of from about 5% to about 30%, preferably from about 10% to about 25%, (and all combinations and subcombinations of ranges therein). In some alternately preferred embodiments, wherein n-butyl acetate is employed, the n-butyl acetate is present at a range of from about 5% to about 15%, more preferably from about 8% to about 12% by weight of the nail coating composition (and all combinations and subcombinations of ranges therein). In yet other aspects, the n-butyl acetate is present at a range of from about 15% to about 30%, more preferably from about 18% to about 28% by weight of the nail coating composition (and all combinations and subcombinations of ranges therein). In some aspects, wherein isobutyl acetate is employed, the isobutyl acetate is present at a range of from about 30% to about 50%, more preferably from about 40% to about 45% by weight of the nail coating composition (and all combinations and subcombinations of ranges therein).

In certain other preferred embodiments, the hybrid nail coating compositions of the present invention include a propyl acetate solvent (for example, n-propyl or isopropyl acetate or any combination thereof). The propyl acetate solvent is typically present at a range of from about 0% to about 20%, preferably from about 1% to about 15%, (and all combinations and subcombinations of ranges therein). In some alternately preferred embodiments, the propyl acetate is present at a range of from about 0% to about 8%, more preferably from about 1% to about 6% by weight of the nail coating composition (and all combinations and subcombinations of ranges therein). In yet other aspects, the propyl acetate is present at a range of from about 1% to about 15%, more preferably from about 2% to about 12% by weight of the nail coating composition (and all combinations and subcombinations of ranges therein).

In certain other preferred embodiments, the hybrid nail coating compositions of the present invention include an ethyl acetate solvent. The ethyl acetate solvent is typically present at a range of from about 15% to about 60%, preferably from about 20% to about 60%, (and all combinations and subcombinations of ranges therein). In some alternately preferred embodiments, the ethyl acetate is present at a range of from about 15% to about 30%, more preferably from about 20% to about 25% by weight of the nail coating composition (and all combinations and subcombinations of ranges therein). In yet other aspects, the ethyl acetate is present at a range of from about 25% to about 60%, more preferably from about 35% to about 60%, still more preferably from about 40% to about 60% by weight of the nail coating composition (and all combinations and subcombinations of ranges therein).

In certain preferred embodiments, the hybrid nail coating compositions of the present invention include ethanol solvent, preferably SD alcohol 40-B, a grade of specifically denatured ethanol, as a solvent in the mixture. The ethanol solvent is typically present at a range from about 2% to about 20%, preferably from about 4% to about 16%, more preferably from about 5% to 15% by weight of the nail coating composition (and all combinations and subcombinations of ranges therein). In some alternately preferred embodiments, the SD alcohol is present at a range of from about 5% to about 10%, or is present at a range of from about 12% to about 16% by weight of the nail coating composition (and all combinations and subcombinations of ranges therein).

In some aspects, the hybrid nail coating compositions of the present invention include a butyl alcohol solvent, including n-butyl, isobutyl, and/or sec-butyl alcohol and mixtures thereof, preferably n-butyl alcohol, as a solvent in the mixture. The butyl alcohol solvent is typically present at a range from about 1% to about 10%, preferably from about 2% to about 5%, more preferably from about 2% to about 4% by weight of the nail coating composition, (and all combinations and subcombinations of ranges therein).

In certain other aspects, the hybrid nail coating compositions of the present invention include isopropyl alcohol as a solvent, which is typically present at a range of from about 1% to about 20%, and preferably present at a level of about 1% to about 15% by weight of the nail coating composition; more preferably from about 3% to about 15%; with from about 8% to about 12% by weight of the nail coating composition being even more preferred (and all combinations and subcombinations of ranges therein). In other alternately preferred embodiments, the isopropyl alcohol is present at a range of from about 1% to about 8%, preferably from about 4% to about 6% by weight of the nail coating composition.

In certain preferred embodiments, the hybrid nail coating compositions of the present invention include diacetone alcohol as a solvent in the mixture, which is typically present at a range of from about 0.06% to about 1%, and preferably present at a level of from about 0.1% to about 0.8% by weight of the nail coating composition (and all combinations and subcombinations of ranges therein). In certain alternately preferred embodiments, diacetone alcohol is present at a range of from about 0.08% to about 0.1% by weight of the nail coating composition.

In certain preferred embodiments, the hybrid nail coating compositions of the present invention include trimethylpentanyl diisobutyrate as a solvent, which is typically present at a range of from about 1% to about 5%, and preferably present at a level of from about 2% to about 5% by weight of the nail coating composition (and all combinations and subcombinations of ranges therein). In certain alternately preferred embodiments, trimethylpentanyl diisobutyrate is present at a range of from about 0.08% to about 0.1% by weight of the nail coating composition.

In certain preferred embodiments, the methyl ethyl ketone (2-butanone) is employed as a solvent, and which is typically present at a range of from about 0.06% to about 3%, and preferably present at a level of from about 1% to about 2% by weight of the nail coating composition (and all combinations and subcombinations of ranges therein).

B. Film Forming Agent

In certain aspects, the hybrid nail coating compositions of the present invention include a film former (i.e., a film forming agent). Film forming agents are typically non-reactive oligomeric or non-reactive polymeric components in their nature, or are chemically or photochemically reactive monomeric or oligomeric components capable of forming a film when acted upon by chemical or photochemical initiators. In some preferred embodiments, the hybrid nail coating compositions of the present invention include one or more film forming agents, preferably two or more agents. In compositions where two or more agents are present, the composition may include both reactive and non-reactive film forming agents. In certain other embodiments where reactive film forming agents are present, the reactive agents may interact with initiators, such as chemical or photochemical initiators, to provide the film forming agent, as may be understood by the ordinarily skilled artisan.

Non-reactive oligomeric or non-reactive polymeric film forming agents include, for example, solvent dissolvable compounds such as nitrocellulose and cellulose esters (e.g., cellulose acetate butyrate, and/or cellulose acetate propionate) hydroxyl ethyl cellulose, hydroxypropyl cellulose, polyvinylbutyral and/or tosylamide formaldehyde resins, polyesters; resins, such as polyurethane resins, alkyd resins, and polyvinyl resins such as polyvinyl acetate, polyvinyl chloride, polyvinylbutyrate; (meth)acrylic and vinyl copolymers such as styrene/butadiene copolymers, acrylate/vinyl acetate copolymers, acrylonitrile/butadiene copolymers, and ethylene/vinyl acetate copolymers. The non-reactive, solvent-dissolvable, film-forming polymer may be a mixture of any acceptable polymeric film forming agent. By way of guidance, the amount of film forming agent or agents in the hybrid nail coating compositions is in the range of from about 4% to about 20% by weight of the composition (and all combinations and subcombinations of ranges therein). As one of ordinary skill in the art would readily understand once armed with the teachings of the present invention, the level in use of the non-reactive film forming agent is somewhat dependent upon the particular film forming agent employed. For example, hydroxylpropyl cellulose requires somewhat higher loadings than nitro cellulose or cellulose esters to achieve a similar effect.

In certain alternate aspects, the reactive film former in compositions or system layers of the present invention comprises monomeric (meth)acrylate esters, for example, monoesters of diols such as ethylene glycol, propane glycol (and the like) and (meth) acrylic acid, preferably methacrylic acid, as well as polyurethane acrylate monomers, oligomers or polymers as described herein. By way of guidance, a reactive film former, when employed, is typically provided at a level of from about 0.05% to about 2% by weight based on the weight of the composition or system layer; preferably from about 0.05% to about 1%, more preferably from about 0.05 to about 0.5%.

Certain embodiments of the hybrid nail coating compositions optionally further include (meth)acrylate monomers or polymers in order to fine tune adhesion and removal properties. Non-limiting examples of such (meth)acrylates include: mono or poly(meth)acrylic acids, HPMA, HEMA, pyromellitic dianhydride di(meth)acrylate, pyromellitic dianhydride glyceryl dimethacrylate, pyromellitic dimethacrylate, methacroyloxyethyl maleate, methacroyloxyethyl succinate, 2-hydroxyethyl methacrylate/succinate, 1,3-glycerol dimethacrylate/succinate adduct, phthalic acid monoethyl methacrylate, ethyl methacrylate, tetrahydrofurfuryl methacrylate, butyl methacrylate, isobutyl methacrylate, PEG-4 dimethacrylate, PPG monomethacrylate, trimethylolpropane trimethacrylate, hydroxyethyl methacrylate, isopropylidenediphenyl bisglycidyl methacrylate, lauryl methacrylate, cyclohexyl methacrylate, hexyl methacrylate, urethane methacrylate, diurethane dimethacrylate, di-HEMA trimethylhexyl dicarbamate, triethylene glycol dimethacrylate, ethylene glycol dimethacrylate, tetraethylene glycol dimethacrylate, trimethylolpropane trimethacrylate, neopentylglycol dimethacylate, acetoacetoxy methacrylate. Di-HEMA trimethylhexyl dicarbamate, as used herein refers to a polyurethane acrylate oligomer (or International Nomenclature of Cosmetic Ingredients (“INCI”) designation, “Di-Hema Trimethylhexyl Dicarbamate”). The polyurethane acrylate oligomer may include monomeric, oligomeric and/or polymeric species, and any combinations thereof.

In certain other preferred embodiments, the hybrid nail coating compositions of the present invention include a dimethicone as a film former in the mixture, which is typically present at a range of from about 0.005% to about 0.02%, more preferably from about 0.005% to about 0.01%, and even more preferably at a level of about 0.0075% by weight of the nail coating composition or system layer. In some alternately preferred embodiments, the dimethicone is present at a range of from about 0.007% to about 0.01% by weight of the nail coating composition or system layer.

C. Photoinitiator

In certain aspects, the hybrid nail coating compositions of the present invention, include a photoinitiator. In other aspects, the hybrid nail coating compositions of the present invention include one or more photoinitiators (“typical photoinitiators”) such as those described herein or as would be understood by one or reasonable skill in the art, alone or in combination with one or more self-initiating oligomers. In other aspects, the hybrid nail coating compositions of the present invention substantially exclude any “typical photoinitiator” as would be understood by one or reasonable skill in the art. As used herein, the substantial absence or substantial exclusion of such photoinitiators refers to a combined weight of any typical photoinitiator(s) in the composition that is less than or equal to 50% by weight of the total weight of photoinitiator in the composition, the total weight inclusive of any self-initiating oligomers present in the formulation. In certain preferred embodiments, the combined weight of any typical photoinitiator(s) in the composition that is less than or equal to 45%, 40%, 35%, 30%, 25%, 20%, 15%, 10%, 5%, 4%, 3%, 2%, or 1% by weight of the total weight of photoinitiator in the composition inclusive of any self-initiating oligomers present in the formulation (and all combinations and subcombinations of ranges therein). In certain alternative aspects, the combined weight of any typical photoinitiator(s) in the composition is less than or equal to 0.5% by weight of the total weight of photoinitiator in the composition. In other alternative aspects, any typical photoinitiator(s) is either present at a de minimus level in the composition or is excluded from the composition altogether.

In preferred embodiments, the photoinitiator comprises a self-initiating oligomer. Use of self-initiating oligomers is reported to eliminate concerns about migration of low molecular weight photoinitiators as well as reduce or eliminate the need for such photoinitiators, because they reportedly generate free radicals upon ultraviolet (“UV”) irradiation without by-product formation and are bound into the matrix of the film upon curing in certain polymer systems. As used herein, the term “self-initiating oligomers” refers to compounds which may photochemically cleave in the presence of UV or visible light wavelengths to provide oligomers bearing a free radical, the radical capable of initiating further oligomerization or polymerization with olefinic compounds present in the composition, or any one or more of uncured coating layers present on the nail at the time of application. Exemplary commercial compounds such as Allnex's brand of EBECRYL® LEO 10101, 10102, and 10103 radiation curing acrylate resins, and Bomar's DYNAX LS® light sensitive aliphatic and aromatic urethane acrylates. Further self-initiating oligomers include compounds where a photoinitiator (preferably Type I) is incorporated into an oligomer backbone. Exemplary self-initiating oligomers include Michael adducts of beta-keto esters and (meth)acrylates and/or (meth)acrylated urethanes. In certain preferred embodiments wherein Michael adducts of beta-keto esters and (meth)acrylates and/or (meth)acrylated urethanes are employed, the self-initiating oligomer comprises a Michael addition adduct of urethane dimethacrylate and an alkyl acetoacetate, such as ethyl acetoacetate. Alternatively, Michael adducts of beta-keto esters and (meth)acrylates and/or (meth)acrylated urethanes may be employed, as self-initiating oligomer wherein the Michael adduct is formed from reaction with monomeric, oligomeric or polymeric forms and mixtures thereof the following: di-HEMA trimethylhexyl dicarbamate, di-HPMA trimethylhexyl dicarbamate; (HEMA)(HPMA) trimethylhexyl dicarbamate, di-HEA trimethylhexyl dicarbamate, di-HPA trimethylhexyl dicarbamate; (HEA)(HPA) trimethylhexyl dicarbamate, di-HEMA isophorone dicarbamate, di-HPMA isophorone dicarbamate; (HEMA)(HPMA) isophorone dicarbamate, di-HEA isophorone dicarbamate, di-HPA isophorone dicarbamate; or (HEA)(HPA) isophorone dicarbamate.

By way of guidance, a self-initiating oligomer compound, when employed, is typically provided at a level of from about 0.05% to about 2% by weight based on the weight of the composition or system layer; preferably from about 0.1% to about 1%, more preferably from about 0.1 to about 0.5%.

D. Plasticizers

In certain preferred embodiments the hybrid nail coating compositions of the present invention include at least one plasticizer. Plasticizers useful in the presently claimed nail enamel composition include plasticizers commonly employed in nail varnish compositions. These plasticizers encompass, but are not limited to, acetyl triethyl citrate, dibutyl phthalate, dioctyl phthalate, tricresyl phthalate, butyl phthalate, dibutoxy ethyl phthalate, diamylphthalate, tosyl amide, N-ethyl-tosyl amide, sucrose acetate isobutyrate, camphor, castor oil, citrate esters, glyceryl diesters, glyceryl triesters, tributyl phosphate, triphenyl phosphate, butyl glycolate, benzyl benzoate, butyl acetyl ricinoleate, butyl stearate, trimethylpentanyl diisobutyrate and dibutyl tartrate.

In some other preferred embodiments, the hybrid nail coating compositions of the present invention include a sucrose acetate butyrate or sucrose acetate isobutyrate as a plasticizer in the mixture, and is typically present at a range of from about 0.01% to about 2%, preferably from about 0.01% to about 1.8%, and even more preferably at a level of about 0.1% to about 1.5% by weight of the nail coating composition (and all combinations and subcombinations of ranges therein).

In some preferred embodiments, the hybrid nail coating compositions of the present invention include camphor as a plasticizer in the mixture. When employed in the compositions of the present invention, camphor is typically present at a range of from about 0.1% to about 2%, and preferably at a level of about 0.3% to about 1.5%, with about 0.5 to about 1% by weight of the nail coating composition being more preferred (and all combinations and subcombinations of ranges therein). In some alternately preferred embodiments, the camphor is present at a range of from about 0.2% to about 0.5%, more preferably from about 0.2% to about 0.4% by weight of the nail coating composition.

In certain other preferred embodiments, the hybrid nail coating compositions of the present invention include triphenyl phosphate as a plasticizer in the mixture, and is typically present at a range of from about 0.2% to about 6%, preferably from about 0.5% to about 5%, and more preferably at a level of from about 2% to about 5% by weight of the nail coating composition (and all combinations and subcombinations of ranges therein).

E. Additional Components

In still other preferred embodiments, the hybrid nail coating compositions of the present invention include a colorant, preferably D&C Violet #2. The D&C Violet #2 is typically present at a range of from about 0.001% to about 0.1%, and even more preferably at a level of about 0.01% by weight of the nail coating composition.

In some preferred embodiments, the hybrid nail coating compositions of the present invention include one or more pigments or dyes that may vary in color that may function as colorants in the mixture, and which may be present at a range of from about 0.01% to about 20%, preferably from about 0.03% to about 18%, and more preferably at a level of from about 0.03% to about 16% by weight of the nail coating composition (and all combinations and subcombinations of ranges therein). Typically, these pigments are present in one or more of the layers of the nail systems of the present invention when color is desired by the consumer. Non-limiting examples of pigments useful in the compositions of the present invention include Titanium Dioxide, Black Iron Oxide, D&C Black #2, FD&C Red #4, D&C Red #6, D&C Red #7, D&C Red #17, D&C Red #21, D&C Red #22, D&C Red #27, D&C Red #28, D&C Red #30, D&C Red #31, D&C Red #33, D&C Red #34, D&C Red #36, D&C Red #40, FD&C Blue #1, D&C Orange #4, D&C Orange #5, D&C Orange #10, D&C Orange #11, D&C Blue #4, D&C Brown #1, FD&C Green #3, D&C Green # 5, D&C Green #6, D&C Green #8, FD&C Yellow #5, FD&C Yellow #6, D&C Yellow #7, D&C Yellow #8, D&C Yellow #10, and D&C Yellow #11 and combinations thereof.

In yet other preferred embodiments, hybrid nail coating compositions of the present invention include a suspending agent or agents in the mixture. In some aspects, the suspending agent comprises stearalkonium hectorite or stearalkonium bentonite preferably stearalkonium hectorite or combination thereof. The suspending agent is typically present at a range of from about 0.1% to about 2%, and preferably at a level of from about 0.2% to about 1.5% by weight of the nail coating composition (and all combinations and subcombinations of ranges therein).

In certain preferred embodiments, the hybrid nail coating compositions of the present invention include a stability enhancer, preferably citric acid, which is typically present at a range of from about 0.005% to about 0.2%, more preferably at a range of from about 0.005% to about 0.1% by weight of the nail coating composition (and all combinations and subcombinations of ranges therein).

In other preferred embodiments, the hybrid nail coating compositions of the present invention further comprise less than about 1% by weight of urethane resin bisphenol. A diglycidyl methacrylate (“BISGMA”, in polymeric, oligomeric and/or monomeric form); more preferably less than about 0.5% of based on the weight of the nail coating composition. In certain other more preferred embodiments, the hybrid nail coating compositions of the present invention do not contain BISGMA urethane resin. The BISGMA based urethane resin is reportedly prepared by reacting the hydroxyl functions of BISGMA with a hydrocarbon diisocyanate. (BISGMA can be purchased from Esstech, and is sold as Nupol 46-4005 from Cook Composites and Polymers.). See Lilley et al., U.S. Pat. No. 6,803,394.

In certain preferred embodiments, the hybrid nail coating compositions of the present invention include a stabilizer such as acrylates copolymer, a general term for copolymers of two or more monomers consisting of acrylic acid, methacrylic acid or one of their simple esters, typically used as a film forming agent, suspending agent as well as an adhesive for nail binding products. It is typically present at a range of from about 1% to about 4%, preferably at a range of from about 2% to about 3% by weight of the nail coating composition (and all combinations and subcombinations of ranges therein).

The compositions and/or system layer compositions according to the invention disclosed herein may also include one or more additives recognized by a person skilled in the art as being capable of incorporation into such nail coating compositions. For example, the composition may include at least one cosmetically active compound, which may be selected from vitamins, minerals, moisturizers, hardening agents such as silica and formaldehyde/glyoxal, UV absorbers (including for example, benzophenone and/or etocrylene), and fibers such as nylon or aramide fibers. Additional additive ingredients may include keratin and its derivatives, melanin, cross linking agents, pH adjusters, collagen, cysteine, chitosan and its derivatives, ceramides, biotin, oligoelements, protein hydrolysates, and phospholipids. Etocrylene and other UV compounds that function as a UV absorber are typically used herein to protect the disclosed compositions from deterioration by UV light. UV absorbers may also assist in reducing the yellowing which is often seen in artificial nails. UV absorbers have the ability to convert incident UV radiation into less damaging infrared radiation (heat), or visible light. A recommended amount of UV absorber is about 0.001% to about 5% by weight of the total composition. Suitable UV absorbers include hydroxy benzotriazole compounds and benzophenone compounds such as are disclosed in U.S. Pat. No. 6,818,207, incorporated herein by reference in its entirety. pH adjusters are optionally employed, in certain aspects, to skew pH, for example, toward a basic pH to facilitate bonding of the second layer composition to the nail surface.

In some preferred embodiments, the nail art paints or nail coating compositions further comprise polyester copolymers, such as those derived from diols, preferably branched alkylene diols, for example, neopentyl glycol, and alkane diacids, such as adipic acid, alkane polyacids, or alkane or aryl acid anhydrides, such as trimellitic anhydride. The polyester copolymer, when employed, is typically present at a range of from about 0.1% to about 10%, preferably at a range of from about 0.5% to about 8% by weight of the nail coating composition (and all combinations and subcombinations of ranges therein).

II. Hybrid Nail Coating Systems

The present invention is also directed, in part, to hybrid nail coating systems comprising a first layer comprising solvent, a film former, and a photoinitiator, the photoinitiator comprising a self-initiating oligomer.

The hybrid nail coating compositions comprising solvent, a film former, and a photoinitiator, the photoinitiator comprising a self-initiating oligomer, may be employed with any base coats, pre-bond compositions color coating and the like or combination thereof, as one of ordinary skill in the art would readily appreciate once armed with the disclosures herein provided. However, it has been advantageously discovered that when the nail coating compositions including self-initiating oligomers are used as a first layer that is applied over an earlier applied second layer composition, as herein described, with or without a third layer composition interposed between the first and second layers, after which the curing is initiated, the resultant nail coating or covering lasts longer or is more durable that typical lacquer nail polishes. Moreover, ease of application, time involved in the application, and radiation exposure are reduced in comparison to gel polishes or acrylics which require curing after each layer is applied to the nail.

Accordingly, in some aspects, the present invention is directed, in part, to hybrid nail coating systems comprising: a first layer comprising solvent, a film former, and a photoinitiator, the photoinitiator comprising a self-initiating oligomer; and a second layer interposed between the first layer and the nail being coated, the second layer comprising a nail adhesion promoting monomer and a solvent.

Typically, the hybrid nail coating composition of the present invention is part of a hybrid nail coating system comprising at least a first layer composition and a second layer composition; the first layer comprising solvent, a film former, and a photoinitiator, the photoinitiator preferably comprising a self-initiating oligomer; and the second layer typically interposed between the first layer and the nail being coated, the second layer comprising a nail adhesion promoting monomer and a solvent. In certain preferred embodiments, a matrix may be formed by associating or reacting a reactive film former with a photoinitiator, preferably formed by associating or reacting a self-initiating oligomer present in the first layer with a nail adhesion promoting monomer in the second layer once both the layers have been applied to the nail and subsequently exposed to UV or visible light. In some preferred embodiments, the association may further confer the second layer monomer's nail adhesion promoting properties across the layer interface between the first and second layers to any polymerizable components contained in the first layer.

In the hybrid nail coating systems of the present invention comprising at least a first layer composition and a second layer composition; the second layer comprises a pre-bond compound, preferably a pre-bond compound structurally containing a nail bonding moiety in one portion of the structure and a reactive olefinic moiety in another portion of the structure. Preferably, these two moieties are positioned structurally at or near opposite termini of the compound, preferably nail adhesion promoting monomer.

The present invention comprises pre-bond compounds that are applied to the natural nail to enhance the adhesive properties of the coating compounds of the invention. In some aspects, the pre-bond compounds typically comprise an acrylate based polymer, preferably an aromatic acid methacrylate, or an olefinically unsaturated carboxylic acid, in a biocompatible solvent. By way of guidance, a pre-bond compound, when employed, is typically provided at a level of from about 0.05% to about 1% by weight based on the weight of the composition or system layer; preferably from about 0.1% to about 0.8%, more preferably from about 0.1% to about 0.5%. An exemplary aromatic acid methacrylate is commercially available as Sarbox SB 500E50 from Sartomer Company located in Exton, Pa. Sarbox SB 500E50 is a proprietary aromatic acid methacrylate half ester provided in ethoxylated trimethylolpropane triacrylate monomer. Alternative pre-bond compounds include, for example, acrylates copolymer, alone or in combination with dimethiconol and/or mercaptopropionic acid. Acrylates copolymer or other pre-bond compound may also be employed, preferentially is employed, in combination with nail adhesion promoting monomer, the adhesion promoting monomer preferably an olefinically unsaturated carboxylic acid capable of free-radically oligomerizing or polymerizing, such as, for example, acryloyloxy ethyl phthalate, methacryloyloxy ethyl phthalate, acryloyloxy ethyl maleate methacryloyloxy ethyl maleate, acryloyloxy ethyl succinate, or methacryloyloxy ethyl succinate, or other olefinically unsaturated carboxylic acid compounds with similar functionality, preferably similar structural functionality. The solvent in the layer is generally biocompatible and evaporates rapidly after being applied to the natural nail. Numerous examples are disclosed hereinabove. It may be a single organic solvent or blend of organic solvents. The solvent is preferably selected from the group consisting of alcohols and esters. In the preferred embodiment, the biocompatible solvent is a mixture of alcohols and esters, preferably selected from the group consisting of C₁-C₄ alkyl acetates, C₁-C₄ alcohols, and trimethylpentanyl diisobutyrate, and any mixture thereof, and comprises between approximately 50% to about 95% by weight and preferably between approximately 60% to about 80% by weight of the composition.

The pre-bond compounds are used in conjunction with the layers associated with hybrid nail coating systems of the present invention to enhance the bonding of the coatings to the natural nail and/or bonding between layers of the inventive systems. As a result, lifting of the coating at either the nail surface in general or the cuticle area, in particular, is reduced. Other pre-bond compounds are disclosed by Patel, U.S. published Application No. 2005/0065297 A1; Montgomery et al. U.S. Pat. No. 4,766,005; Steffier, U.S. Pat. No. 5,965,147; Schoon et al., U.S. Pat. No. 8,481,010 B2; Pagano et al., U.S. Pat. No. 5,772,988 and Eppinger, EP 0 453 628 A2, the disclosures of each of which are hereby incorporated herein by reference in their entireties.

Typically, the second layer composition comprising an adhesion promoting compound or nail adhesion promoting monomer is applied to a nail and allowed to evaporate solvent, thereby forming a film on the nail, before the first layer composition comprising a solvent, a film former, and a photoinitiator, or an optional third layer (interposed between the first layer and the second layer, the third layer comprising a solvent, a film former, a (meth)acrylated urethane, and pigments or colorants) is applied. Preferably, a (meth)acrylated urethane is incorporated in the third layer composition. While the second layer comprising a nail adhesion promoting monomer may be partially cured after its application to the nail, it is more typically allowed to form a film without any substantial curing (somewhat analogous to prior art nail lacquer applications). While not wishing to be held to any theory or theories of operation, it is believed that omitting a curing step at this point allows for the association and/or formation of a matrix between first and second layers (and optionally interposed third layer), after application of the first layer, and subsequent system activation by UV or visible light.

As used herein, the term “partially cured” refers to less than about 90% of the available nail adhesion promoting monomer present in the second layer being cured before application of the first layer. In certain preferred aspects, the term refers to a curing of less than or equal to 80%, 70%, 60%, 50%, 40%, 30%, 20%, 10%, 5%, 4%, 3%, 2%, or 1% of the available nail adhesion promoting monomer present in the second layer. In alternately preferred aspects, only adventitious curing occurs, or no purposeful curing is carried out prior to application of a layer comprising solvent, a film former and a photoinitiator, preferably one or more self-initiating oligomer(s) (“first layer”). Generally speaking, adventitious, premature, or otherwise undesired curing can be reduced, minimized or substantially eliminated by the conscious exclusion of any photoinitiator in the layer comprising a nail adhesion promoting monomer.

As used herein, the term “substantial curing” refers to greater than or equal to 50% of the available reactive olefins (monomer, oligomer, or polymer) in a layer being further reacted or cured after contact to free-radical initiators as a consequence of exposure photochemical or chemical means for initiation. In certain aspects, the wt. % amount of reactive monomeric, oligomeric, or polymeric olefins present in the compositions or system layers substantially cured is greater than or equal to 60%, 70%, 80%, 90%, 95%, 96%, 97%, 98%, or 99% of the available reactive olefin present in the system layer or composition. In alternately preferred aspects, only a de minimus amount of reactive olefins the system layer or composition remains unreacted post curing.

In addition to a pre-bond compound, preferably nail adhesion promoting monomer, and biocompatible solvent, the second layer composition may further comprise at least one of a film forming agent (preferably a non-reactive film forming agent), pigment, plasticizer, stabilizer, cosmetically active compound, polyester copolymer, or any combination thereof, wherein each is as described hereinabove.

In other aspects, the present invention is directed, in part, to hybrid nail coating systems comprising: a first layer comprising a solvent, a film former, and a photoinitiator, the photoinitiator comprising a self-initiating oligomer; a second layer interposed between the first layer and the nail being coated, the second layer comprising a nail adhesion promoting monomer and a solvent; and a third layer interposed between the first layer and the second layer, the third layer comprising solvent, a film former, and a (meth)acrylated urethane.

In certain aspects, the hybrid nail coating systems further comprise a third layer composition, the third layer interposed between the first layer composition and the second layer composition, and comprising a solvent, a film former, and a (meth)acrylated urethane. A (meth)acrylated urethane is typically present in the third layer composition at a level in a range of from about 0.05% to about 0.5% by weight of the third layer composition (and all combinations and subcombinations thereof). In certain aspects it is present at a level in a range of from about 0.05% to about 0.25% by weight of the third layer composition.

Exemplary third layer composition solvents include esters and alcohols and their mixtures, such as those disclosed hereinabove for layers or compositions of the present invention. Alternatively, the solvents include ethyl acetate, propyl acetate, butyl acetate, isopropanol, ethanol, butanol, diacetone alcohol, and trimethylpentanyl- disiobutyrate, and mixtures thereof. It may be a single organic solvent or blend of organic solvents. The solvent is preferably selected from the group consisting of alcohols and esters. In the preferred embodiment, the biocompatible solvent is a mixture of alcohols and esters, preferably selected from the group consisting of C₁-C₄ alkyl acetates, C₁-C₄ alcohols, and trimethylpentanyl diisobutyrate, and any mixture thereof, and comprises between approximately 50% to about 95% by weight and preferably between approximately 55% to about 80% by weight, of the composition (and all combinations and subcombinations thereof).

In addition to solvent, a film former, and a (meth)acrylated urethane, preferably diurethane dimethacrylate or polyurethane acrylate oligomer as described herein, the third layer composition may further comprise at least one of a pigment or colorant; plasticizer; stabilizer; stability enhancer; cosmetically active compound; and polyester copolymer; or any combination thereof, wherein each is as described hereinabove.

In certain other embodiments, the compositions of the present invention may be cured by any process with components that may be incorporated into the composition and which provides a free radical source capable of curing the nail coating compositions, so long as the resultant compositions may be safely employed and applied. This includes, for example, any thermochemically or photochemically induced free radical processes known to the ordinarily skilled artisan as well as those employing catalysts to initiate the generation of free radicals and, hence, the curing of the nail coating compositions.

III. Kits Containing Hybrid Nail Coating Compositions/Systems

In certain other embodiments, the present invention is directed to kits containing hybrid nail coating compositions or hybrid nail coating systems that contain such hybrid nail coating compositions, the hybrid nail coating compositions comprising: a solvent, a film former, and a photoinitiator, the photoinitiator comprising a self-initiating oligomer; wherein the kit comprises the nail coating composition or system containing such hybrid nail coating compositions; and a bottle for containing hybrid nail coating compositions, the bottle designed to substantially exclude the passage of UV and/or visible light.

IV. Methods of Use

In some embodiments, the present invention is directed to methods of coating mammalian nails with a hybrid nail coating composition, wherein the method comprises: applying a nail coating composition according to the present invention contiguously to a mammalian nail or previously applied nail coating composition; and curing the composition on the nail or nail coating.

In some embodiments, the present invention is directed to methods of coating mammalian nails with a hybrid nail coating composition, wherein the method comprises: applying a nail coating composition according to the present invention contiguously to a mammalian nail or previously applied nail coating composition; and curing the composition on the nail or nail coating.

In other embodiments, the present invention is directed to methods of coating mammalian nails with a hybrid nail coating system, wherein the method comprises: applying a second layer composition according to the present invention contiguously to a mammalian nail; thereafter applying a first layer composition according to the present invention contiguously to a mammalian nail; and thereafter curing the composition on the nail or nail coating.

In other embodiments, the present invention is directed to methods of coating mammalian nails with a hybrid nail coating system, wherein the method comprises: applying a second layer composition according to the present invention contiguously to a mammalian nail; thereafter applying a first layer composition according to the present invention contiguously to a mammalian nail; and thereafter curing the composition on the nail or nail coating; wherein no substantial curing takes place between application of the second layer and application of the first layer.

In other embodiments, the present invention is directed to methods of coating mammalian nails with a hybrid nail coating system, wherein the method comprises: applying a second layer composition according to the present invention contiguously to a mammalian nail; thereafter applying a third layer composition according to the present invention contiguously to a mammalian nail; thereafter applying a first layer composition according to the present invention contiguously to a mammalian nail; and thereafter curing the composition on the nail or nail coating.

In some preferred embodiments, typical compositions and/or systems of the present invention comprise at least a first layer composition that is activated and cured under UV or ambient light.

Any UV single or multiple light emitting source is contemplated herein by the inventor. The UV light source is not critical so long as the light source is a UV spectrum range light emitter and the power of such single or multiple light source is sufficient to activate and/or harden (i.e., cure) the nail coating composition in a desirable time. Typical lights may include UV light bulb sources and/or light emitting diode (“LED”) lights, or any other equivalent light source, or any combination thereof. Typical lights may include UV light bulb sources and/or light emitting diode (“LED”) lights, or any other equivalent light source, or any combination thereof. UV radiation may be characterized by a wavelength, or group of wavelengths, typically, but not limited to about 320 to about 420 nanometers. Adventitious room illumination, visible light wavelengths or sunlight may also be used to cure the composition or system of layers.

In other embodiments, the compositions of the present invention may be applied in analogous fashion to typical prior art polishes, such as by brush application.

In other embodiments, the compositions, kits containing such compositions and/or systems including the compositions, and or methods employing such compositions, systems and/or kits of the present invention provide coatings that may provide a harder and/or less brittle finish, more durability, and/or better nail coverage than prior art lacquer compositions or systems.

In certain embodiments involving actual uses, the nail is prepared for the hybrid nail coating and a thin layer of the pre-bond material, preferably a second layer of the present invention, is applied. A tacky surface may result to which the hybrid composition (first layer composition of the present invention) or alternatively, a third layer composition is applied, followed by a first layer composition, each according to the present invention.

In other embodiments of the present invention, the compositions and/or kits containing such compositions are provided as a bottle application, and in yet other embodiments of the present invention the compositions and/or kits containing such compositions are provided as a brush application.

The compositions, systems, kits containing such compositions, and methods of use for such compositions, systems and/or kits of the present invention may offer other further advantages as compared to currently available lacquers or ultraviolet-cured gel products that are currently available. For example, in some embodiments of the present invention, the compositions are contained in bottles designed to substantially exclude UV and/or visible light to deter activation of the formula by outside light during storage. The materials used to construct bottles designed to hold the nail coating compositions of the present invention may inherently exclude such light in certain embodiments. Alternately, other bottles not possessing these characteristics, including, for example, clear bottles, may be finished or coated with, for example, one or more special UV-protective or visible light coatings, including clear coatings. Either of these bottle alternatives may enable more of the nail gel to be used by the end user, for example, by reducing the level of inadvertent activation of the gel contained in the bottle prior to its application. In some instances this may allow substantially all of the product to be used for its intended purpose. A further advantage of providing the compositions in a clear-coated bottle is that the ultimate customer may then have a more ready ability to see and/or select the desired gel nail coating composition color. This is contrasted to existing ultraviolet-cured gel products that are traditionally provided in an opaquely colored white or black jar, which denies the customer an ability to see the actual color of the nail coating composition. In other embodiments of the present invention, the compositions may provide nail coatings that are longer lasting, more durable and more resistant to chipping, especially heavy chipping than prior art lacquer products. In yet other embodiments, the compositions, systems, kits and/or methods of the present invention are more readily and/or easily applied and/or with less UV exposure to skin than prior art gel polishes.

In some preferred embodiments, the hybrid nail coating compositions of the present invention include one or more cross-linking agents. Typically, these cross-linking agents are esters of a polyhydroxy compound and methacrylic acid, wherein a substantial number of the hydroxy groups of the polyhydroxy compound, and preferably each of the hydroxy groups, have been esterified with methacrylic acid. The polyhydroxy compounds preferably have 3 or more hydroxy groups per molecule, more preferably 3 or 4 hydroxy groups, still more preferably 3 hydroxy groups per molecule of polyhydroxy compound. In certain yet more preferred embodiments, the cross-linking agent is trimethylolpropane trimethacrylate (“TMPTA”). The cross-linking agent is typically present at a range of from about 0.02% to about 0.35%, more preferably from about 0.02% to about 0.3%, still more preferably from about 0.02% to about 0.2% by weight of the nail coating composition.

In yet other preferred embodiments, hybrid nail coating compositions and/or system layers of the present invention include a suspending agent, preferably stearalkonium hectorite or stearalkonium bentonite, or any combination thereof. When used in combination, the ratio of a suspending agent to one or more other suspending agents in the composition or system layer will typically depend upon, inter alia, the physical properties and components comprising the layer or composition. The total amount of suspending agent present in the layer or composition is generally in a range of from about 0.2% to about 2%, and preferably at a level of about 0.5% to about 2% by weight of the nail coating composition or system layer.

In still other preferred embodiments, the hybrid nail coating compositions of the present invention further comprise one or more additives, wherein the additive(s) are other than maleimide functional materials, such as for example, hydroxy ethylmaleimide, triethylene glycol biscarbonate bisethylmaleimide, 2-isopropyl urethane ethylmaleimide, 2-acryloyl ethylmaleimide, acetoxy ethyl maleimide, isophorone bisurethane bisethylmaleimide, N,N′-hexamethylenebismaleimide, and/or N,N′-(2,2,4trimethylhexamethylene)-bismaleimide.

In certain aspects, the compositions and/or the system layers of the present invention further comprise less than about 1% by weight of hydroxypropyl methacrylacrylic acid (“HPMA”); preferably less than about 0.5% of based on the weight of the nail coating composition or system layer. In certain other more preferred embodiments, the hybrid nail coating compositions of the present invention do not contain HPMA.

In certain aspects, the compositions and/or the system layers of the present invention further comprise less than about 1% by weight of hydroxyethyl methacrylacrylic acid (“HEMA”); preferably less than about 0.5% of based on the weight of the nail coating composition or system layer. In certain other more preferred embodiments, the hybrid nail coating compositions of the present invention do not contain HEMA. In certain aspects, the compositions and/or the system layers of the present invention further comprise urethane(methacrylates). In other aspects, one or two of the system layers comprise urethane(methacrylates). In yet other aspects, the second layer does not comprise more than de minimus quantities of urethane(methacrylates). In still other aspects, the urethane(methacrylates) are present in greater than de minimus quantities only in the color layer (third layer compositions of the present invention).

In some embodiments, the base coatings (second layer coating compositions) have the following formulations, Formulations 1 to 11 shown in Table 1:

TABLE 1
BASE COATS

| | 1 | 2 | 3 | 4 | 5 | 6 | 7 | 8 | 9 | 10 | 11 |
|---|---|---|---|---|---|---|---|---|---|---|---|
| Ingredients | Wt % | Wt % | Wt % | Wt % | Wt % | Wt % | Wt % | Wt % | Wt % | Wt % | Wt % |
| Ethyl Acetate | 28 | 28 | 28 | 28 | 28 | 28 | 28 | 28 | 28 | 28 | 28 |
| Butyl Acetate | 25 | 25 | 25 | 25 | 25 | 25 | 25 | 25 | 25 | 25 | 25 |
| SD Alcohol 40B | 15 | 15 | 15 | 15 | 15 | 15 | 15 | 15 | 15 | 15 | 15 |
| Nitrocellulose | 11.35 | 11.45 | 11.3 | 11.35 | 11.4 | 11.25 | 11.35 | 11.34 | 11.35 | 11.45 | 11.3 |
| Adipic Acid/Neoopentyl | 5.6 | 5.6 | 5.6 | 5.6 | 5.6 | 5.6 | 5.6 | 5.6 | 5.6 | 5.6 | 5.6 |
| Glycol/Trimellitic Anhydride | | | | | | | | | | | |
| Copolymer | | | | | | | | | | | |
| Isopropyl Alcohol | 5 | 5 | 5 | 5 | 5 | 5 | 5 | 5 | 5 | 5 | 5 |
| Triphenyl Phosphate | 3.5 | 3.5 | 3.5 | 3.5 | 3.5 | 3.5 | 3.5 | 3.5 | 3.5 | 3.5 | 3.5 |
| Trimethyl Pentanyl | 3.5 | 3.5 | 3.5 | 3.5 | 3.5 | 3.5 | 3.5 | 3.5 | 3.5 | 3.5 | 3.5 |
| Diisobutyrate | | | | | | | | | | | |
| Sucrose Acetate Isobutyrate | 1.35 | 1.35 | 1.35 | 1.35 | 1.35 | 1.35 | 1.35 | 1.35 | 1.35 | 1.35 | 1.35 |
| Etocrylene | 1 | 1 | 1 | 1 | 1 | 1 | 1 | 1 | 1 | 1 | 1 |
| Acrylates Copolymer (and) | 0.5 | 0.5 | 0.5 | 0.5 | 0.5 | 0.5 | 0.5 | 0.5 | 0.5 | 0.5 | 0.5 |
| Dimethiconol (and) | | | | | | | | | | | |
| Mercaptopropionic | | | | | | | | | | | |
| Acid | | | | | | | | | | | |
| Acryloyloxy ethyl phthalate | 0.2 | 0.1 | 0.25 | | | | 0.1 | 0.2 | 0.2 | 0.1 | 0.25 |
| Methacryloyloxy ethyl | | | | 0.2 | 0.15 | | 0.1 | | | | |
| maleate | | | | | | | | | | | |
| Methacryloyloxy ethyl | | | | | | 0.3 | | | | | |
| succinate | | | | | | | | | | | |
| Dimethicone | | | | | | | | 0.005 | | | |
| Titanium Dioxide | | | | | | | | 0.0026 | | | |
| Tocophenyl Acetate | | | | | | | | 0.001 | | | |
| Hydrolized Corn Protein & | | | | | | | | 0.001 | | | |
| Hydrolyzed Wheat Protein & | | | | | | | | | | | |
| Hydrolyzed | | | | | | | | | | | |
| Soy Protein & | | | | | | | | | | | |
| Leuconostac/Radish Root | | | | | | | | | | | |
| Ferment Filtrate | | | | | | | | | | | |

In other embodiments, the compositions (first layer coating compositions) have the following formulations, Formulations 12 to 32 shown in Table 2:

TABLE 2

| | TOP COATS | | | | | | | | | | | |
|---|---|---|---|---|---|---|---|---|---|---|---|---|
| | 12 | 13 | 14 | 15 | 16 | 17 | 18 | 19 | 20 | 21 | 22 | 23 |
| Ingredients | Wt % | Wt % | Wt % | Wt % | Wt % | Wt % | Wt % | Wt % | Wt % | Wt % | Wt % | Wt % |
| Ethyl Acetate | 55 | 55 | 55 | 23.4 | 23.4 | 23.4 | 23.4 | 23.4 | 23.4 | 56.09 | 56.09 | 56.09 |
| Isopropyl | 15 | 15 | 15 | 8 | 8 | 8 | 8 | 8 | 8 | 3.5 | 3.45 | 3.35 |
| Alcohol | | | | | | | | | | | | |
| Cellulose | 10 | 10 | 10 | | | | | | | | | |
| Acetate | | | | | | | | | | | | |
| Butyrate | | | | | | | | | | | | |
| Butyl Acetate | 10 | 10 | 10 | | | | | | | | | |
| Propyl Acetate | 5.849 | 5.799 | 5.699 | | | | | | | | | |
| Acrylates | 2 | 2 | 2 | | | | | | | | | |
| Copolymer | | | | | | | | | | | | |
| Triphenyl | 1 | 1 | 1 | | | | | | | | | |
| Phosphate | | | | | | | | | | | | |
| Sucrose | 1 | 1 | 1 | | | | | | | | | |
| Benzoate | | | | | | | | | | | | |
| Benzophenone-1 | 0.1 | 0.1 | 0.1 | 0.09 | 0.09 | 0.09 | 0.09 | 0.09 | 0.09 | | | |
| Michael Adduct | 0.05 | 0.1 | 0.2 | 0.05 | 0.1 | 0.2 | | | | 0.05 | 0.1 | 0.2 |
| of Urethane | | | | | | | | | | | | |
| Dimethacrylate | | | | | | | | | | | | |
| & Ethyl | | | | | | | | | | | | |
| Violet 2 | 0.001 | 0.001 | 0.001 | 0.01 | 0.01 | 0.01 | 0.01 | 0.01 | 0.01 | | | |
| Isobutyl Acetate | | | | 49.2 | 49.2 | 49.2 | 49.2 | 49.2 | 49.2 | | | |
| Hydroxy Propoly | | | | 17.3 | 17.3 | 17.3 | 17.3 | 17.3 | 17.3 | | | |
| Cellulose | | | | | | | | | | | | |
| MEK | | | | 1.95 | 1.9 | 1.75 | 1.95 | 1.9 | 1.75 | | | |
| Michael Adduct | | | | | | | 0.05 | 0.1 | 0.2 | | | |
| of Tripropylene | | | | | | | | | | | | |
| glycol | | | | | | | | | | | | |
| dimethacrylate | | | | | | | | | | | | |
| & Ethyl | | | | | | | | | | | | |
| Acetoacetate | | | | | | | | | | | | |
| Nitrocellulose | | | | | | | | | | 8 | 8 | 8 |
| Hydrated Silica | | | | | | | | | | 5.5 | 5.5 | 5.5 |
| Trimethyl | | | | | | | | | | 4.73 | 4.73 | 4.73 |
| Pentanyl | | | | | | | | | | | | |
| Diisobutyrate | | | | | | | | | | | | |
| Adipic Aced/ | | | | | | | | | | 4.03 | 4.03 | 4.03 |
| Neopentyl | | | | | | | | | | | | |
| Glycol/ | | | | | | | | | | | | |
| Trimellitic | | | | | | | | | | | | |
| Anhydride | | | | | | | | | | | | |
| Copolymer | | | | | | | | | | | | |
| Stearalkonium | | | | | | | | | | 1.29 | 1.29 | 1.29 |
| Hectorite | | | | | | | | | | | | |
| Citric Acid | | | | | | | | | | 0.01 | 0.01 | 0.01 |
| Acetyl Triethyl | | | | | | | | | | | | |
| Citrate | | | | | | | | | | | | |
| Ebecryl LEO | | | | | | | | | | | | |
| 10101 | | | | | | | | | | | | |
| (from Allnex) | | | | | | | | | | | | |
| Benzophenone-1 | | | | | | | | | | | | |
| Ebecryl LEO | | | | | | | | | | | | |
| 10102 | | | | | | | | | | | | |
| (from Allnex) | | | | | | | | | | | | |
| | | | | 24 | 25 | 26 | 27 | 28 | 29 | 30 | 31 | 32 |

| Ingredients | Wt % | Wt % | Wt % | Wt % | Wt % | Wt % | Wt % | Wt % | Wt % |
|---|---|---|---|---|---|---|---|---|---|
| Ethyl Acetate | 56.09 | 56.09 | 56.09 | 42.3803 | 42.4222 | 42.322 | 42.3803 | 42.4222 | 42.3222 |
| Isopropyl | 3.5 | 3.5 | 3.5 | 11.0061 | 11.0061 | 11.006 | 11.0061 | 11.0061 | 11.0061 |
| Alcohol | | | | | | | | | |
| Cellulose | | | | 6.0132 | 6.0132 | 6.0132 | 6.0132 | 6.0132 | 6.0132 |
| Acetate | | | | | | | | | |
| Butyrate | | | | | | | | | |
| Butyl Acetate | 16.8 | 16.8 | 16.8 | 18.7545 | 18.7545 | 18.755 | 18.7545 | 18.7545 | 18.7545 |
| Propyl Acetate | | | | 2.4053 | 2.4053 | 2.4053 | 2.4053 | 2.4053 | 2.4053 |
| Acrylates | | | | 1.3995 | 1.3995 | 1.3995 | 1.3995 | 1.3995 | 1.3995 |
| Copolymer | | | | | | | | | |
| Triphenyl | | | | 0.6013 | 0.6013 | 0.6013 | 0.6013 | 0.6013 | 0.6013 |
| Phosphate | | | | | | | | | |
| Sucrose | | | | 0.5211 | 0.5211 | 0.5211 | 0.5211 | 0.5211 | 0.5211 |
| Benzoate | | | | | | | | | |
| Benzophenone-1 | | | | | | | | | |
| Michael Adduct | | | | | | | | | |
| of Urethane | | | | | | | | | |
| Dimethacrylate | | | | | | | | | |
| & Ethyl | | | | | | | | | |
| Violet 2 | | | | 0.001 | 0.001 | 0.001 | 0.001 | 0.001 | 0.001 |
| Isobutyl Acetate | | | | | | | | | |
| Hydroxy Propoly | | | | | | | | | |
| Cellulose | | | | | | | | | |
| MEK | | | | | | | | | |
| Michael Adduct | 0.05 | 0.1 | 0.2 | | | | | | |
| of Tripropylene | | | | | | | | | |
| glycol | | | | | | | | | |
| dimethacrylate | | | | | | | | | |
| & Ethyl | | | | | | | | | |
| Acetoacetate | | | | | | | | | |
| Nitrocellulose | 8 | | | 5.977 | 5.977 | 5.977 | 5.977 | 5.977 | 5.977 |
| Hydrated Silica | 5.5 | | | | | | | | |
| Trimethyl | 4.73 | | | | | | | | |
| Pentanyl | | | | | | | | | |
| Diisobutyrate | | | | | | | | | |
| Adipic Aced/ | 4.03 | | | 5.9771 | 5.9771 | 5.9771 | 5.9771 | 5.9771 | 5.9771 |
| Neopentyl | | | | | | | | | |
| Glycol/ | | | | | | | | | |
| Trimellitic | | | | | | | | | |
| Anhydride | | | | | | | | | |
| Copolymer | | | | | | | | | |
| Stearalkonium | 1.29 | | | | | | | | |
| Hectorite | | | | | | | | | |
| Citric Acid | 0.01 | | | | | | | | |
| Acetyl Triethyl | | | | 4.7816 | 4.7816 | 4.7816 | 4.7816 | 4.7816 | 4.7816 |
| Citrate | | | | | | | | | |
| Ebecryl LEO | | | | 0.1419 | 0.1419 | 0.1419 | 0.1419 | 0.1419 | 0.1419 |
| 10101 | | | | | | | | | |
| (from Allnex) | | | | | | | | | |
| Benzophenone-1 | | | | 0.0401 | 0.0401 | 0.0401 | 0.0401 | 0.0401 | 0.0401 |
| Ebecryl LEO | | | | | | | 0.1419 | 0.1 | 0.2 |
| 10102 | | | | | | | | | |
| (from Allnex) | | | | | | | | | |

In some embodiments, the interposed third layer compositions (shown with optional pigments herein) of the present invention have the following formulations, Formulations 33 to 44 show in Table 3:

TABLE 3
COLOR COATS

| | 33 | 34 | 35 | 36 | 37 | 38 | 39 | 40 | 41 | 42 | 43 | 44 |
|---|---|---|---|---|---|---|---|---|---|---|---|---|
| Ingredients | Wt % | Wt % | Wt % | Wt % | Wt % | Wt % | Wt % | Wt % | Wt % | Wt % | Wt % | Wt % |
| Ethyl Acetate | 23.88 | 23.883 | 23.88 | 26.79 | 26.79 | 26.79 | 23.5 | 23.5 | 23.5 | 23.9 | 23.88 | 23.88 |
| Butyl Acetate | 22.7 | 22.7 | 22.7 | 25.45 | 25.45 | 25.45 | 19.5 | 19.5 | 19.5 | 22.7 | 22.7 | 22.7 |
| Nitrocellulose | 11.9 | 11.9 | 11.9 | 11.1 | 11.1 | 11.1 | 11.8 | 11.8 | 11.8 | 11.9 | 11.9 | 11.9 |
| Propyl Acetate | 9.5 | 9.5 | 9.5 | 3.9 | 3.9 | 3.9 | | | | 9.5 | 9.5 | 9.5 |
| Tosylamide Formaldehyde | 9.1 | 9.1 | 9.1 | | | | | | | 9.1 | 9.1 | 9.1 |
| Isopropyl Alcohol | 5.5 | 5.5 | 5.5 | 5.1 | 5.1 | 5.1 | 4.95 | 4.95 | 4.95 | 5.5 | 5.5 | 5.5 |
| Triphenyl Phosphate | 4.3 | 4.3 | 4.3 | 4 | 4 | 4 | 3.8 | 3.8 | 3.8 | 4.3 | 4.3 | 4.3 |
| Trinethyl Pentanyl | 4.3 | 4.3 | 4.3 | 4.5 | 4.5 | 4.5 | 3.8 | 3.8 | 3.8 | 4.3 | 4.3 | 4.3 |
| Stearalkonium Bentonite | 1 | 1 | 1 | 0.9 | 0.9 | 0.9 | | | | 1 | 1 | 1 |
| Camphor | 0.9 | 0.8 | 0.7 | | | | | | | 0.8 | 0.9 | 0.7 |
| Sucrose Acetate Isobutyrate | 0.5 | 0.5 | 0.5 | 0.2 | 0.2 | 0.2 | | | | 0.5 | 0.5 | 0.5 |
| Adipic Acid/Neopentyl | 0.5 | 0.5 | 0.5 | 8 | 8 | 8 | 4.2 | 4.1 | 4.2 | 0.5 | 0.5 | 0.5 |
| Glycol/Trimellitic Anhydride | | | | | | | | | | | | |
| Stearalkonium Hectorite | 0.2 | 0.2 | 0.2 | 0.3 | 0.25 | 0.3 | | | | 0.2 | 0.2 | 0.2 |
| Diurethane Dimethacrylate | 0.05 | 0.1 | 0.2 | 0.05 | 0.1 | 0.2 | 0.05 | 0.1 | 0.2 | | | |
| Diacetone Alcohol | 0.1 | 0.1 | 0.1 | 0.79 | 0.79 | 0.64 | | | | 0.1 | 0.1 | 0.1 |
| Citric Acid | 0.1 | 0.1 | 0.1 | 0.02 | 0.02 | 0.02 | | | | 0.1 | 0.1 | 0.1 |
| Benzophenone-1 | 0.05 | 0.05 | 0.05 | 0.05 | 0.05 | 0.05 | | | | 0.05 | 0.05 | 0.05 |
| Dimethicone | 0.005 | 0.005 | 0.005 | 0.005 | 0.005 | 0.005 | | | | 0.01 | 0.005 | 0.005 |
| Pigments | 5.412 | 5.412 | 5.412 | 7 | 7 | 7 | | | | 5.41 | 5.412 | 5.412 |
| n-Butyl Alcohol | | | | 1.9 | 1.9 | 1.9 | | | | | | |
| SD Alcohol 40B | | | | | | | 7.6 | 7.6 | 7.6 | | | |
| Acrylates Copolymer | | | | | | | 2.4 | 2.4 | 2.4 | | | |
| Silica | | | | | | | 2.4 | 2.4 | 2.2 | | | |
| Violet 2 | | | | | | | 0 | 0 | 0 | | | |
| Pigments | | | | | | | 16 | 16 | 16 | | | |
| Di-Hema Trimethylhexyl | | | | | | | | | | 0.1 | 0.05 | 0.2 |

A person skilled in the art can, without undue experimentation, select those optional additional compounds and/or their quantity, so that the advantageous properties of the composition according to the invention are not, or are not substantially, impaired by the inclusion of such additives.

The compositions according to the invention may be prepared by a person skilled in the art on the basis of his or her general knowledge and according to the state of the art.

The compositions according to the invention are also useful in the kits and or methods of use of the present invention.

The invention will be further clarified by the following examples, which are intended to be illustrative of the invention, but not limiting thereof.

### EXAMPLES

The following procedure sets out a typical method for applying the systems of the present invention. After application of a base coat (second layer composition as described herein), 1 or 2 coats of colored coat (third layer composition as described herein), and a coat of top coat (first layer as described herein) are applied in that order. Photoscission of self-initiating oligomer contained in the first layer composition takes place when this system is exposed to ambient light, and curing of the first layer is initiated leading to a cured nail coating system.

To support and demonstrate the improved performance of the systems of the present invention, a blind test is conducted on volunteers to compare a standard lacquer nail polish system with a hybrid system of the invention. Standard lacquer system layers lack self-initiating oligomers, reactive film forming agents and/or nail adhesion promoting monomers but are otherwise analogously formulated. Four different shades of hybrid and conventional lacquers are applied to the nails of 5 volunteers for a total of 20 volunteers. The varied shades of a conventional lacquer system and exemplary hybrid lacquer system are each applied on alternate nails of a volunteer and are monitored at fixed intervals. A rating scale developed for indication of wear to the coating is as follows. Wear (most desired) is better than slight chipping which is better than chipping which is better than heavy chipping (least desired). Day 5 wear is observed and visually evaluated. The qualitative results are plotted in the chart. The X axis shows the degree of wear and the Y-axis shows the number of digits (nails). Wear for conventional lacquers is compared with wear for hybrid lacquers in the Day 5 Hybrid Lacquer Chart shown in Table 4. At end of Day 5, 60 digits show only wear (no chipping observed) for the exemplary hybrid lacquer while conventional lacquer shows only wear on 65 digits. Hybrid lacquer systems (blue bar graph) show less extensive chipping than conventional systems (red bar graph). For exemplary hybrid systems, 16 digits show slight chipping, 4 digits show chipping, and no heavy chipping is observed as compared with tested conventional lacquer systems in which 12 digits show slight chipping, 24 digits showed chipping, and 4 show heavy chipping. The data show that the tested conventional system shows more extensive chipping that the hybrid system of the present invention. When queried, each volunteer indicates on average that they would wear hybrid nail composition of the present invention for at least 2 days longer than the conventional system before the coating became too unsightly and requires its removal.

The gloss measurements of the hybrid nail coating system and certain prior art nail lacquer coatings are carried out as follows on Leneta Drawdown Charts. A 6 mil wet film of base coat is drawn and allowed to dry for about two hours. A color coating (color coat of conventional and hybrid nail coating third layer) was made on top of the base coat (hybrid nail coating second layer) with a 6 mil coating tool. The color coat is allowed to dry for about an hour and then a top coat for conventional lacquer system and hybrid nail coating system (hybrid nail coating first layer) is drawn over the combined base coat/color coat with a 6 mil coating tool. The system is dried overnight and gloss readings are taken using a glossmeter. The conventional lacquer system had gloss of 93.3 units as compared with the hybrid nail coating system gloss of 98.6 units when analyzed at a 65 degree angle.

When any variable occurs more than one time in any formula, its definition in each occurrence is independent of its definition at every other occurrence. Combinations of variables are permissible only if such combinations result in stable formulations.

It is believed the chemical formulas and names used herein correctly and accurately reflect the underlying chemical compounds. However, the nature and value of the present invention does not depend upon the theoretical correctness of these formulae, in whole or in part. Thus it is understood that the formulas used herein, as well as the chemical names attributed to the correspondingly indicated compounds, are not intended to limit the invention in any way.

When ranges are used herein for physical properties, elements or variables in formula compositions, per cent composition of elements or variables in formulas, all combinations and subcombinations of ranges and specific embodiments therein are intended to be included. Unless otherwise indicated, % ranges for components are expressed on a weight per total weight basis. For example, if ethyl acetate is used as a solvent at 10%, then the ethyl acetate weight added is equal to 10% of the total weight of the composition or system layer formulation.

The disclosures of each patent, patent application and publication cited or described in this document are hereby incorporated herein by reference, in their entirety.

The invention illustratively disclosed herein suitably may be practiced in the absence of any element which is not specifically disclosed herein. The invention illustratively disclosed herein suitably may also be practiced in the absence of any element which is not specifically disclosed herein and that does not materially affect the basic and novel characteristics of the claimed invention.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A hybrid nail coating system comprising: a first layer comprising a solvent, a film former, and a photoinitiator, the photoinitiator comprising a self-initiating oligomer.

2. The hybrid nail coating system according to claim 1, wherein the self-initiating oligomer comprises a moiety selected from the group consisting of EBECRYL® LEO 10101, EBECRYL® LEO 10102, and EBECRYL® LEO 10103 radiation curing acrylate resins; DYNAX LS® light sensitive aliphatic and aromatic urethane acrylates; Michael adducts of beta-keto esters and (meth)acrylates and/or (meth)acrylated urethanes; and combinations thereof.

3. The hybrid nail coating system of claim 1, further comprising a second layer interposed between the first layer and a nail being coated, the second layer comprising a nail adhesion promoting monomer and a solvent.

4. The hybrid nail coating system according to claim 3, wherein the nail adhesion promoting monomer comprises an olefinically unsaturated carboxylic acid capable of free radically polymerizing.

5. The hybrid nail coating system according to claim 3, wherein the nail adhesion promoting monomer comprises acryloyloxyethyl phthalate, methacryloyloxyethyl maleate, or methacryloyloxyethyl succinate.

6. The hybrid nail coating system according to claim 3, further comprising a third layer interposed between the first layer and the second layer, the third layer comprising a solvent, a film former, a (meth)acrylated urethane, and a pigment or colorant.

7. The hybrid nail coating system according to claim 6, wherein the (meth)acrylated urethane comprises diurethane dimethacrylate or di-HEMA trimethylhexyl dicarbamate.

8. The hybrid nail coating system according to claim 1, wherein the coating is cured with UV or ambient light.

9. A hybrid nail coating composition comprising: a solvent, a film former, and a photoinitiator, the photoinitiator comprising a self-initiating oligomer.

10. A kit suitable for coating mammalian nails comprising a hybrid nail coating composition or hybrid nail coating system that contains the hybrid nail coating composition, the hybrid nail coating composition comprising:
solvent, a film former, and a photoinitiator, the photoinitiator comprising a self-initiating oligomer;
wherein the kit comprises the nail coating composition or system containing such hybrid nail coating compositions; and a bottle for containing hybrid nail coating compositions, the bottle designed to substantially exclude the passage of UV and/or visible light.

11. A method for coating mammalian nails with a hybrid nail coating system, wherein the method comprises:
applying a second layer composition contiguously to a mammalian nail, the second layer composition comprising a nail adhesion promoting monomer and a solvent;
thereafter, applying a first layer composition contiguously to a mammalian nail, the first layer composition comprising a solvent, a film former, and a photoinitiator, the photoinitiator comprising a self-initiating oligomer; and
thereafter, curing the compositions on the nail or nail coating.

12. The method according to claim 11, wherein the method further comprises applying a third layer composition contiguously to a mammalian nail after the application of the second layer and prior to the application of the first layer, the third layer composition comprising a solvent, a film former, a (meth)acrylated urethane, and a pigment or colorant.

13. The method according to claim 11, wherein the system is cured with UV or ambient light.
